(19) United States
(12) Patent Application Publication
(10) Pub. No.: US 2016/0270641 A1
(43) Pub. Date: Sep. 22, 2016

(54) VIDEO ASSISTED SURGICAL DEVICE

(71) Applicant: A.M. SURGICAL, INC., Smithtown, NY (US)

(72) Inventors: Romi Mirza, Smithtown, NY (US); Ather Mirza, Smithtown, NY (US)

(21) Appl. No.: 14/961,537

(22) Filed: Dec. 7, 2015

Related U.S. Application Data
(60) Provisional application No. 62/134,914, filed on Mar. 18, 2015.

(51) Int. Cl. A61B 1/045; A61B 1/06; A61B 1/00; A61B 17/32; A61B 10/04; A61B 10/02

(52) U.S. Cl. CPC A61B 1/045; A61B 10/04; A61B 10/025; A61B 1/00016; A61B 17/320016; A61B 1/0684; A61B 2018/00595

(57) ABSTRACT

The present application discloses a disposable video-assisted surgical device for performing endoscopic and arthroscopic surgery on any target tissue, bone, joint or target area of the body of a subject. The application also discloses a kit having the video-assisted surgical device. A method for using the video-assisted surgical device in endoscopic and arthroscopic surgery is also described and is applicable to any target tissue, bone, joint or target area of the body of a subject.

This application claims priority to Provisional U.S. Patent Application No. 62/134,914, filed on Mar. 18, 2015. The entirety of the aforementioned application is incorporated herein by reference.

### FIELD

This application generally relates to medical devices. In particular, the application relates to video assisted surgical devices and the use thereof.

### BACKGROUND

Conventional surgical techniques and equipment often require a fairly large incision over the surgical site and spreading of the incision to allow viewing and instrument access. These techniques can require a longer period of recovery than endoscopic methods and have greater levels of post-operative pain due to the incision size and level of manipulation during the procedure.

Endoscopic and arthroscopic surgeries are minimally invasive surgical procedures that are performed through small incisions or natural body openings. An endoscopic or arthroscopic procedure typically involves use of specialized devices and direct- or remote-control manipulation of instruments with indirect observation of the surgical field through an endoscope, arthroscope or similar device. Compared to open surgery, endoscopic and arthroscopic surgery is a minimally invasive surgery with less postoperative pain, early resumption of usual activities and a cosmetically appealing scar. It typically results in shorter hospital stays, or allows outpatient treatment.

In general, arthroscopy is applied to introduction of a scope into a joint anywhere in the body. Arthroscopic surgery refers to the process of introducing of the instrument to, and performing an operation at the joint. Endoscopy is applied to introduction of a scope into a body cavity anywhere in the body. Endoscopic surgery refers to the process of introducing the instruments and performing surgery at the operation site. Further nomenclature is designated by the anatomical structure the scope is introduced into, for example if the scope is placed in the stomach it is called Gastroscopy, in the abdomen it is Laprascopy, etc. There are places where no actual cavity exists. Here, surgeons can create a cavity by introducing a slotted cannula to visualize the surroundings without soft tissue obstruction, as in endoscopic carpal tunnel and cubital tunnel.

As seen in recent outbreaks of infections in hospitals, the insufficient or improper sterilization of re-usable surgical implements, can result in the introduction of microorganisms, including drug-resistant bacteria, into the patient, potentially resulting in severe, or even lethal, infections. This risk is magnified in procedures that require the insertion of multiple instruments into an incision.

The present application fulfills a need in the art for a compact, disposable device for uniportal endoscopic and arthroscopic surgery that comprises all of the individual tools needed, including a camera, following the creation of an entry portal. The device and method of the present application allow the insertion of a single member through the entry portal, with all of the individual tools contained therewithin, allowing that member to remain in situ throughout the procedure, thereby eliminating the need to insert and withdraw multiple instruments through the entry portal. The entire device can then be disposed of as medical waste following the procedure, thereby eliminating the need for re-sterilization and minimizing the threat of patient infection. Additionally, the present device comprises integrated camera and lighting, allowing wireless real-time transmission of images to any viewing device, providing the advantage of allowing small clinics or individual practitioners to provide endoscopic services without the need to invest in costly equipment that can also be expensive to maintain.

### SUMMARY

One aspect of the present application related to a device for wireless endoscopic and arthroscopic surgery. The device comprises a housing comprising a receptacle for a cannula; a camera with a lens; a control board having transmitter and receiver functions for wirelessly communicating with at least one external receiver/transmitter; an antenna; and a power source. The camera, the control board, the antenna and the power source are enclosed by the housing. In some embodiments, the device can be handheld. In some further embodiments, the housing is formed in a shape to facilitate gripping with a single hand. The device can be used on any target tissue, bone, joint or target area of the body of a subject.

Another aspect of the present application relates to a wireless device for performing video-assisted endoscopic and arthroscopic surgical procedures, comprising: a cannula having a tubular body with a proximal end, a distal end and an open slot on the tubular body; a camera with a lens; a light source that illuminates into and through the cannula; a control board having transmitter and receiver functions for wirelessly communicating with at least one external receiver/transmitter; an antenna; and a power source, wherein the light source, the control board and the antenna are mounted inside an enclosed housing, wherein the housing has a distal end and a proximal end and wherein the cannula is attached to the distal end of the housing. In some embodiments, the device can be handheld. In some further embodiments, the housing is formed in a shape to facilitate gripping with a single hand. The device can be used on any target tissue, bone, joint or target area of the body of a subject.

Yet another aspect of the present application relates to a system for wireless endoscopic and arthroscopic surgery. The system comprises a device for wireless endoscopic and arthroscopic surgery that comprises a housing comprising a receptacle for a cannula; a camera with a lens; a control board having transmitter and receiver functions for wirelessly communicating with at least one external receiver/transmitter; an antenna; and a power source, wherein the camera, the control board, the antenna and the power source are enclosed by the housing. The system further comprises an external receiver/transmitter for visualizing images transmitted by the device. In some embodiments, the device can be handheld. In some further embodiments, the housing is formed in a shape to facilitate gripping with a single hand. The device and system can be used on any target tissue, bone, joint or target area of the body of a subject.

A further aspect of the present application relates to a method for performing a uniportal endoscopic or arthroscopic procedure with a wireless device for performing video-assisted endoscopic and arthroscopic surgical procedures, the device comprising: a cannula having a tubular body with a proximal end, a distal end and an open slot on the tubular body; a camera with a lens; a light source that illuminates into and through the cannula; a control board having transmitter and receiver functions for wirelessly communicating with at least one external receiver/transmitter; an antenna; and a power source, wherein the light source, the control board and the antenna are mounted inside an enclosed housing, wherein the housing has a distal end and a proximal end and wherein the cannula is attached to the distal end of the housing. In some embodiments, the device can be handheld. In some further embodiments, the housing is formed in a shape to facilitate gripping with a single hand. The device can be used on any target tissue, bone, joint or target area of the body of a subject. The method comprises the steps of a) establishing an entry portal; b) inserting the distal end of the cannula through the entry portal; c) advancing the cannula to create a passage to the target tissue; d) imaging the target tissue with the camera; e) performing an endoscopic or arthroscopic operation at the target tissue; and f) withdrawing the cannula from the entry portal.

Still another aspect of the present application relates to a kit for performing an endoscopic or arthroscopic procedure. The kit comprises a device for wireless endoscopic and arthroscopic surgery that comprises a housing comprising a receptacle for a cannula; a camera with a lens; a control board having transmitter and receiver functions for wirelessly communicating with at least one external receiver/transmitter; an antenna; and a power source, wherein the camera, the control board, the antenna and the power source are enclosed by the housing. The kit further comprises a clear cannula mountable to the device. In some embodiments, the device can be handheld. In some further embodiments, the housing is formed in a shape to facilitate gripping with a single hand. The device can be used on any target tissue, bone, joint or target area of the body of a subject.

### BRIEF DESCRIPTION OF THE DRAWINGS

The present application can be better understood by reference to the following drawings. The drawings are merely exemplary to illustrate certain features that may be used singularly or in any combination with other features and the present application should not be limited to the embodiments shown.

FIG. 1 shows an exemplary embodiment of the present application.

FIGS. 2A-C show top (2A), side (2B) and distal end (2C) perspective views of an exemplary embodiment of the present invention.

### DETAILED DESCRIPTION

The following detailed description is presented to enable any person skilled in the art to make and use the object of this application. For purposes of explanation, specific nomenclature is set forth to provide a thorough understanding of the present application. However, it will be apparent to one skilled in the art that these specific details are not required to practice the subject of this application. Descriptions of specific applications are provided only as representative examples. The present application is not intended to be limited to the embodiments shown, but is to be accorded the widest possible scope consistent with the principles and features disclosed herein.

This description is intended to be read in connection with the accompanying drawings, which are to be considered part of the entire written description of this application. The drawing figures are not necessarily to scale and certain features of the application may be shown exaggerated in scale or in somewhat schematic form in the interest of clarity and conciseness. In the description, relative terms such as “front,” “back,” “up,” “down,” “top,” “bottom,” “upper,” “lower,” “distal,” and “proximal” as well as derivatives thereof, should be construed to refer to the orientation as then described or as shown in the drawing figure under discussion. These relative terms are for convenience of description and normally are not intended to require a particular orientation. Terms concerning attachments, coupling and the like, such as “connected,” “mounted,” and “attached,” refer to a relationship wherein structures are secured or attached to one another either directly or indirectly through intervening structures, as well as both movable or rigid attachments or relationships, unless expressly described otherwise.

The term “trigger finger,” as used herein, also refers to “trigger digit,” “trigger thumb,” and “stenosing tendovaginitis.”

As used herein, the terms “horizontal” and “vertical,” and derivatives of those terms, are used in respect to their relationship to the plane defined by the slot in the cannula of the present application. “Vertical” refers to the plane that can, for example, pass through the slot of the cannula and bisect the cannula into two equal halves, while “horizontal” refers to a plane that is perpendicular to the vertical plane. The horizontal plane may be a level plane with respect to the length of the cannula or housing of the device, or may be at an angle to that level plane, allowing some upward or downward movement of elements moving along the horizontal plane with respect to the level plane.

As used herein, the term “subject” refers to an animal. In some embodiments, the animal is a mammal. In further embodiments, the mammal is a human.

The present application describes a disposable, compact device for performing uniportal endoscopic and arthroscopic surgical procedures. In some embodiments, the device comprises a rigid or flexible cannula, dependent upon the procedure for which the device is to be used, that is attached to the distal end of a housing. In some further embodiments, the cannula is clear, being made of a transparent material. In some embodiments, the procedure can be a uniportal percutaneous endoscopic surgical procedure.

In some embodiments, the cannula has an open proximal end where it is attached to the distal end of the housing, such that surgical tools stored within the housing can be extended into the cannula. In some embodiments, the cannula comprises a closed distal end. In some further embodiments, the closed distal end of the cannula is upturned and comprises an edge for separating, but not cutting, tissues as the cannula is advanced from an entry portal towards a target tissue. In some embodiments, the cannula comprises a longitudinal slot that extends from the proximate end to the proximity of the distal end of the cannula. In some further embodiments, the distal end of the slot is contiguous with an open distal end of the cannula. In other further embodiments, the distal end of the slot is closed. In still other embodiments, the cannula comprises open proximate and distal ends, with the longitudinal surfaces of the cannula being closed.

The housing further comprises a blade and camera that can be extended into the cannula. Particular embodiments of the present device also include a probe and/or cautery within the housing and that can be extended into the cannula.

Additional embodiments of the present invention may include any type of tool that would be useful for a general or specific endoscopic or arthroscopic surgical procedure, such as a rasp for clearing synovium or implements for harvesting a section of tissue for biopsy, grafting or ex vivo growth. The device also comprises a mechanism for advancing/withdrawing the aforementioned tools into/from the cannula. Tools such as the blade, probe and cautery are advanced into the cannula along with the camera in order to perform a desired procedure. The tool protrudes vertically through the slot of the cannula in order to contact a target tissue and perform the desired procedure, while under the observation of the camera, which remains within the lumen of the cannula. Additionally, the housing further comprises a light source for illuminating said procedure, a circuit board for controlling the functions of the device, a wireless transmitter/receiver/antenna assembly for communicating between the device and a remote control panel or video monitor. The housing further contains a power source, such as a battery. The preassembled nature of the device also provides convenience for the practitioner in that the cannula, camera and tools are available in a single package that requires no further assembly and can be used easily in an office setting without the need for some traditional endoscopic or arthroscopic equipment that may be too expensive or cumbersome to use outside of a hospital. Additionally, the present device also can be easily transported and used in remote settings, such as by emergency medical personnel, first responders or military medical personnel.

The use of the present device is exemplified in this application for, but not limited to, endoscopic surgical division of a pulley or tunnel. Some other non-limiting uses for the present device include, for example, other divisions or partial separation of a tendon or ligament, cutting, dividing, separating or making an incision in connective tissue, muscle, cartilage, membranes, skin, other body tissues or organs or any other use of the device that can be envisioned or carried out by the practitioner. As used herein, the term “practitioner” refers to one of skill in the art or any other user of the present device. In some embodiments, the device can be used for a uniportal endoscopic viewing and/or surgical procedure. In other embodiments, the device can be used for an arthroscopic, laparoscopic, or thoracoscopic viewing and/or surgical procedure. As used herein, “laparoscopic” and “thoracoscopic” procedures fall within the scope of “endoscopic” and “arthroscopic” procedures.

Endoscopic or arthroscopic surgical procedures that can be performed with a cannula or device of the present application include, but are not limited to, carpal tunnel release, Guyon's canal (or tunnel) release, cubital tunnel release, plantar fascia release, lateral release for patella realignment, release of radial tunnel, release of pronatar teres, release of trigger finger, release of lacertus fibrosus, tendon release, release of the extensor tendons for lateral epicondylitis, release of medial epicondylitis, release of the posterior and other compartments of the leg, forearm fascia release for fascial compartment syndrome, release of fascial compartments in the upper or lower extremities, relieving the compression of a nerve by a ligament pulley or tunnel, and releasing the travel of a ligament or tendon through a pulley or tunnel. Procedures that can be performed with a cannula or device of the present application include endoscopic or arthroscopic surgical procedures on the spine, such as discectomy for the treatment of degenerative disc disease, herniated discs, bulging discs, pinched nerves or sciatica. Procedures that can be performed with a cannula or device of the present application also include procedures on cranial and facial tissues, as well as fasciotomy release throughout the body. The cannula or device of the present application can be used for blood vessel, including vein or artery, harvesting throughout the body, for example to provide blood vessel graft material in conjunction with a coronary bypass procedure or for a reconstructive surgical procedure. Procedures that can be performed with a cannula or device of the present application also include endoscopic procedures on the wrist and hand, including the palmar and dorsal sides of the hand. Endoscopic procedures that can be performed with a cannula or device of the present application on the hand also include the digits, including the thumb, index finger, middle finger, ring finger and little (pinky) finger. Other examples of endoscopic or arthroscopic procedures that can be performed with a device of the present application include, but are not limited to, observation of internal tissues or injuries, cauterization of vessels, harvesting of tissues for ex vivo growth; obtaining biopsies; spinal surgery; endonasal surgery; mucosal resection; removal of parasites, cysts or tumors, and foreign body retrieval. Still other examples of endoscopic or arthroscopic surgery that can be performed with the device include, but are not limited to, procedures on or within bone, in or around joints or the tendons associated with those joints, as well as any tissue, area or cavity of the body of a subject.

In some embodiments, the present device can be used in the head of a subject. Exemplary procedures in the head include, but are not limited to, nasal surgery, endoscopic sinus surgery, endoscopic pituitary surgery, cranial surgery, endoscopic ear surgery, throat surgery, endodontic surgery and tonsils.

In some embodiments, the present device can be used in the neck of a subject. Exemplary procedures in the neck include, but are not limited to, laryngoscopic surgery, vocal cord surgery, esophageal surgery, thyroid surgery, carotid artery surgery, and brachial plexus surgery.

In some embodiments, the present device can be used in the chest of a subject. Exemplary procedures in the chest include, but are not limited to, endoscopic mediastinal surgery, thoracic surgery, heart surgery, esophageal surgery, and upper gastrointestinal (GI) scoping.

In some embodiments, the present device can be used in a procedure of a finger, hand, foot of a subject.

In some other embodiments, the present device can be used in the abdomen of a subject. Exemplary procedures in the abdomen include, but are not limited to, diagnostic laparoscopy, laparoscopic gastric surgery, laparoscopic liver surgery, laparoscopic pancreatic surgery, laparoscopic nephrectomy and kidney surgery, laparoscopic intestinal surgery, laparoscopic oophorectomy, laparoscopic hysterectomy, laparoscopic urinary bladder surgery, laparoscopic prostate surgery, laparoscopic aortic surgery, laparoscopic appendectomy, laparoscopic colon surgery, endoscopic hysterotomy, endoscopic fetal surgery, endoscopic hernia repair, and endoscopic splenectomy.

In some embodiments, the present device can be used in an upper extremity of a subject. Exemplary procedures in an upper extremity include, but are not limited to, ECTR, ECUTR, endoscopic pronator teres release, forearm fascial compartment release, endoscopic repair of biceps tendon, endoscopic release of lateral and medial epicondylitis, endoscopic release of radial tunnel syndrome, endoscopic surgery of the brachial plexus, endoscopic harvesting of nerve graft, arthroscopy and surgery of wrist, arthroscopy of elbow, arthroscopy and surgery of the carpometacarpal (CMC) joint, arthroscopy and surgery of shoulder, arthroscopy and surgery of acromioclavicular (AC) joint.

In some embodiments, the present device can be used in a lower extremity of a subject. Exemplary procedures in an lower extremity include, but are not limited to, femoral artery surgery, fascia lata release, knee lateral release, endoscopic peroneal nerve release, endoscopic leg fascial compartment release, endoscopic release of gastrocnemius, endoscopic tarsal tunnel release, endoscopic release of Morton's neuroma, endoscopic release of the plantar fascia, arthroscopy of hip, knee and ankle, subtalar joint, and endoscopic harvesting of nerve and tendon graft.

Endoscopic or arthroscopic surgical procedures that can be performed with a device of the present application, such as, but not limited to, a ligament or fascia release procedure, can be performed by approaching the target tissue through an incision or body opening on either the proximate or distal side of the target tissue.

In some embodiments, a device of the present application can be used for plastic surgery. A device of the present application is useful for tissue remodeling or the excision of tissue segments, including necrotic tissue.

One aspect of the present application relates to a wireless device for performing video-assisted surgical procedures comprising a clear cannula having a proximate end and a distal end, further having a slot on one surface; and a grip-able housing having a proximate end and a distal end, wherein the proximate end of the cannula attaches to the distal end of the housing, wherein the housing internally comprises a probe that is extendable into the cannula and capable of protruding through the slot to contact tissue, a blade that is extendable into the cannula and capable of protruding through the slot to contact tissue, a cautery that is extendable into the cannula and capable of protruding through the slot to contact tissue, a camera, a light source that illuminates into and through the cannula, an internal control board having transmitter and receiver functions for wirelessly communicating with at least one external receiver and transmitter, an internal antenna, and a power source.

In one embodiment, the slot is located near the distal end of the cannula. In some further embodiments, the blade is held stationary in relation to the cannula and the cannula is moved in the proximate direction to divide a target tissue. In other further embodiments, the protruding blade is held stationary in relation to the cannula and the cannula is moved in the distal direction to divide a target tissue.

In another embodiment, the slot extends from the proximate end of the cannula to the proximity of the distal end of the cannula. In some further embodiments, the protruding blade slides along the slot in a proximate to distal direction to divide a target tissue. In other further embodiments, the protruding blade slides along the slot in a distal to proximate direction to divide a target tissue.

In some embodiments, the distal end of the cannula is open and the slot is continuous with that open distal end.

In other embodiments, the distal end of the cannula is closed. In further embodiments, the closed distal end of the cannula is pointed.

In other further embodiments, the closed distal end of the cannula comprises a edge for separating tissues. In further embodiments, the edge can be sharpened or non-sharpened.

In some embodiments, the distal end of the cannula is turned upwards like an obturator.

In particular embodiments, the extension of the probe, blade, or cautery is controlled remotely.

In other particular embodiments, the camera views the tissues surrounding the cannula. In further embodiments, the camera is extendable into the cannula along with the probe, blade, or cautery. In still further embodiments, the camera views the probe, camera, or cautery. In other still further embodiments, the extension of the camera is controlled remotely.

In particular embodiments, the view of the camera can be rotated.

In some embodiments, the camera lens has a diameter of less than about 5 mm. In still other embodiments, the camera lens has a diameter of less than about 4 mm. In yet other embodiments, the camera lens has a diameter of less than about 3 mm. In even other embodiments, the camera lens has a diameter of less than about 2 mm.

In other particular embodiments, the camera is a NANEYE camera.

In some embodiments, the camera lens is an optical fiber lens.

In some embodiments, the light source is an LED light source.

In some embodiments, the light source has a diameter of less than about 5 mm. In still other embodiments, the light source has a diameter of less than about 4 mm. In yet other embodiments, the light source has a diameter of less than about 3 mm. In even other embodiments, the light source has a diameter of less than about 2 mm.

In some embodiments, the light source is attached to a fiber optic cable for transmitting light in the cannula. In some further embodiments, the fiber optic cable is embedded into a wall of the cannula. In some embodiments, the fiber optic cable illuminates the entire length of the cannula.

In still other embodiments, the cannula is expandable laterally to spread tissue.

In yet other embodiments, the power source is a battery.

In some particular embodiments, the device is single-use and disposable.

In some embodiments, the device comprises an information display provided in the housing to display frequency information of a transmission frequency of the wireless transmitter. In further embodiments, the device comprises an information reading device for reading the frequency information of the information display.

In some embodiments, the internal control board having transmitter and receiver functions for wirelessly communicating and the at least one external receiver and transmitter communicate via radio frequency communication signals (e.g., FM radio signal). In other embodiments, communication is via microwave or infrared (IR) communication signals from the wireless sensor. In still other embodiments, communication is via short-wavelength radio transmissions, for example in the ISM band from 2400-2480 MHz (IEEE 802.15.1, or Bluetooth).

In some embodiments, the external receiver and transmitter is interfaced with a computer terminal. In some further embodiments, the computer terminal is a notebook computer. In other embodiments, the external receiver and transmitter is interfaced with a tablet or smart phone.

In some embodiments, each of said tools is individually associated with an independent camera operatively attached to that tool.

In other embodiments, the housing further comprises a video display showing real time images from a camera of the device.

In some embodiments, the device comprises non-volatile memory for storing images or information from a performed procedure, wherein said images or information can be retrieved from the device. In some embodiments, the non-volatile memory is an RFID tag. In other embodiments, the non-volatile memory is a micro-SD card.

In some embodiments, the device could be attached to, held, by, inserted into or under the control of a robotic device or tool.

In some embodiments, the device can be directly controlled such as, but not limited to, manual control. In other embodiments, the device can be remotely controlled.

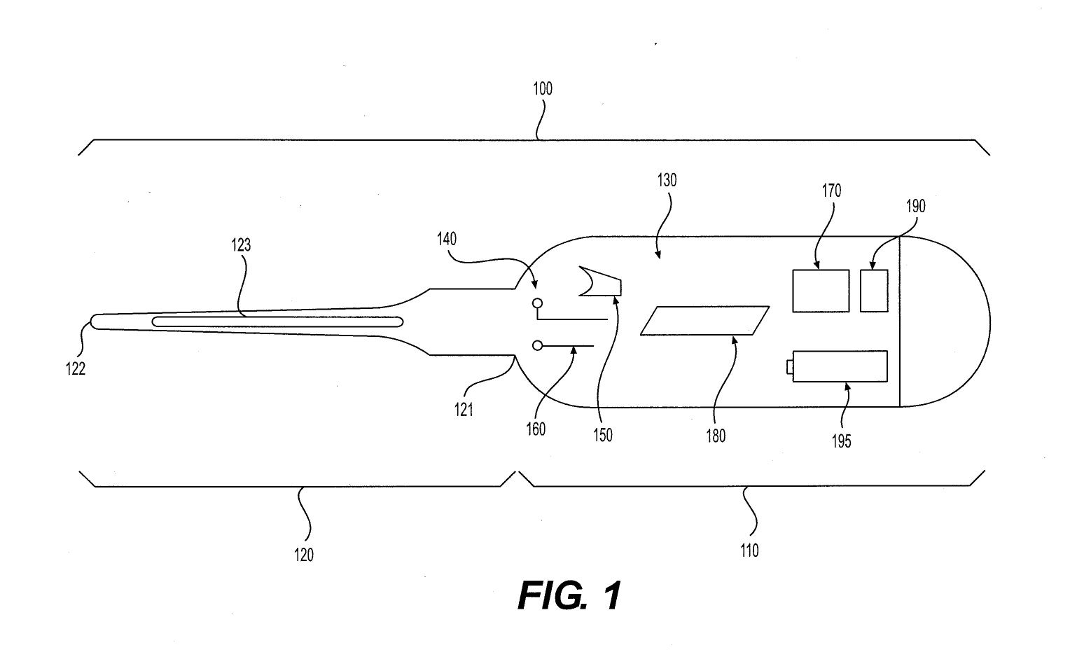

FIG. 1 shows one exemplary embodiment of the device 100 of the present application. The device generally comprises a housing 110 and a cannula 120. The cannula 120 comprises an open central lumen, a proximal end 121 and a distal end 122, wherein the proximal end 121 of the cannula 120 is attached to the distal end of the housing 110.

In some embodiments, the cannula 120 is composed of a clear material. In further embodiments, the clear material is polycarbonate. In some embodiments, the cannula is marked with gradations showing how far the cannula 120 had been inserted through an entry portal.

The cannula 120 of the present device 100 comprises a slot 123 in its upper surface, wherein said slot 123 is contiguous with the open central lumen. In some embodiments, said upper surface is flattened, in other embodiments, said upper surface is rounded. In some embodiments, the slot 123 extends from the proximal end 121 to the proximity of the distal end 122 of the cannula 120. In other embodiments, the slot extends from a point located between the proximal end 121 and distal end 122 to the proximity of the distal end 122. As used herein, “the proximity of the distal end” has the meaning of the slot 123 ending prior to actually joining the distal end 122, i.e., having at least a minimal bridge of material crossing between the distal end of the slot 123 and the distal end 122 of the cannula 120 to prevent over-advancement of a deployed tool through the distal end 122 of the cannula 120.

In other embodiments, the distal end of the slot 123 is contiguous with an open distal end 122 of the cannula 120.

In some embodiments, the distal end 122 of the cannula 120 is closed, as an obturator. In some embodiments, said distal end 122 is pointed. In other embodiments, the distal end 122 comprises a leading edge that is turned upwards, allowing the cannula to separate and form a passage from the entry portal through/between/under/over body tissues to and/or past a target tissue. In some further embodiments, the edge can be flattened.

In some embodiments, the body of the cannula 120 is laterally expandable in order to spread tissue as a passage is made by the cannula 120, obviating the need for inserting a separate instrument through the entry portal to spread tissue.

Also as shown in FIG. 1, the camera 130 and surgical tools are contained within the housing 110 of the device 100 prior to deployment into the cannula 120.

The device 100 comprises a camera 130 that is small enough to deploy into the central lumen of the cannula 120. The camera 130 is generally a high resolution camera, but is at least of sufficient resolution for imaging with sufficient clarity to distinguish different bodily tissues from one another and to image a target tissue with sufficient clarity in order to observe the performance of a surgical procedure on the target tissue. The camera 130 can be advanced into the cannula 120 independently of any surgical tools in order to image/observe bodily tissues or target tissue surrounding the cannula 120 or through the slot 123 before, after or in lieu of a surgical procedure. Having an integral camera 130 within the device, eliminates the need to insert a separate endoscopic camera into the device or an entry portal, thereby eliminating the need for another separate element in the procedure.

The camera 130 can also be advanced into the cannula 120 in association with the probe 140, blade 150, cautery 160 or other suitable surgical tool. In general, the camera comprises within its field of view any portion of the probe 140, blade 150, cautery 160 or other suitable surgical tool that is in contact with, or performing a desired surgical procedure on, a target or bodily tissue. In some embodiments, the camera is a NANEYE camera. In other embodiments, the camera 130 has a resolution of at least 100×100 pixels. In a further embodiment, the camera 130 has a resolution of at least 150×150 pixels. In a still further embodiment, the camera 130 has a resolution of at least 200×200 pixels. In an even further embodiment, the camera 130 has a resolution of at least 250×250 pixels. In some embodiments, there is a separate camera 130 independently associated with each tool of the device 100.

In another embodiment, the camera 130 remains in a fixed position within the housing 110. In a further embodiment, the camera 130 comprises an image transmitting optical fiber, which is attached at its proximal end to the camera. In some still further embodiments, the distal end of the image transmitting optical fiber is movable and moves into the cannula 120 independently or with tools of the device 100. In other still further embodiments, the distal end of the image transmitting optical fiber is in a fixed position in the proximity of the distal end 122 of the cannula, such that from the fixed position of the image transmitting optical fiber the camera 130 can observe and image the surgical procedure.

In still another embodiment, the device 100 comprises a combination of at least one movable camera 130 and at least one fixed position camera 130 as described above. In some embodiments, a camera 130 of the present device 100 comprises a camera body and a lens assembly that is attached to the camera body via an image transmitting optical fiber.

Still referring to FIG. 1, in some embodiments, the device comprises a probe element 140. The probe 140 can be advanced into the cannula 120 and protrudes vertically through the slot 123 in order to, for example, move tissues above the slot 123, provide a reference point for imaging, determine the edges of the target tissue or remove synovium from the target tissue.

Also referring to FIG. 1, the device 100 comprises a blade 150 for performing surgical procedures on a target tissue. The blade 150 can be advanced into the cannula 120 and protrudes vertically through the slot 123 in order to divide a target tissue. In some embodiments, the blade 150 comprises at least one cutting surface on its distal side and division of the target tissue is performed by moving the blade through the slot 123 in a proximal 121 to distal 122 direction. In other embodiments, the blade 150 comprises at least one cutting surface on its proximal side and division of the target tissue is performed by moving the blade through the slot 123 in a distal 122 to proximal 121 direction.

FIG. 1 also shows an embodiment of the device 100 comprising a cautery element 160. The cautery 160 can be advanced into the cannula 120 and protrudes vertically through the slot 123 in order to cauterize a target tissue. In some embodiments, the target tissue was previously divided with the blade 150 of the device 100 during the same surgical procedure. In another embodiment, the target tissue was previously divided by a blade in an earlier surgical procedure. In yet another embodiment, the target tissue was in need of cauterizing due to an earlier injury or other outstanding medical condition.

In some embodiments, the device 100 further comprises a light source 170 contained within the housing 110. The light source 170 provides illumination for the camera 130 in order to allow visualization of bodily or target tissues through the cannula 120 or slot 123. In some embodiments, the position of the light source 170 is fixed within the housing 110 of the device 100. In other embodiments, the light source 170 is associated with the camera 130 and travels with the camera 130, either into the cannula 120, or staying within the housing 110, but moving closer to the proximal end 121 of the cannula 120 as the camera 130 is advanced toward the distal end 122 of the cannula 120. In still other embodiments, the light source 170 comprises a main body whose position is fixed within the housing and is attached to the proximal end of a light transmitting fiber, the distal end of which provides light for the camera 130. In some further embodiments, the distal end of the light transmitting fiber moves in concert with the camera 130. In other further embodiments, the distal end of the light transmitting fiber remains in a fixed position within the cannula 120, for example, in the proximity of the distal end 122 of the cannula. In some embodiments, the light source 170 is a semiconductor light source. In some embodiments, the light source 170 is a light emitting diode (LED) light source. In some embodiments, the device 100 comprises a plurality of light sources 170 as described above that can be in a fixed position and/or moveable.

Still in FIG. 1, in some embodiments, the device 100 comprises a circuit board 180 for processing imagery obtained by the camera 130. Said imagery is transmitted to a remote control or video display via a wireless antenna 190 contained within the housing 110 of the device 100. In some instances, the circuit board receives instructions from the remote control via the wireless antenna 190. In some embodiments, the movement of the camera 130 and the tools of the device 100 are controlled remotely via instruction transmitted to the circuit board 180. In other embodiments, the housing 110 of the device 100 comprises manual control for selecting tools and/or advancing/withdrawing tools or the camera 130 into/from the cannula 120.

Also depicted in FIG. 1, the device 100 further comprises an integral power source 195 to provide energy for the camera 130, light source 170, circuit board 180 and any mechanical functions within the device 100. In some embodiments, the power source 195 is a battery. In further embodiments, the battery is a lithium battery. In some embodiments, the power source 195 is installed within the device 100 upon manufacture, or prior to provision to a practitioner. In other embodiments, the power source 195 is provided separately from the device 100 and installed into the device 100 prior to the use of the device 100 in a surgical procedure. In some embodiments, the power source 195 is removable from the device 100 for separate disposal.

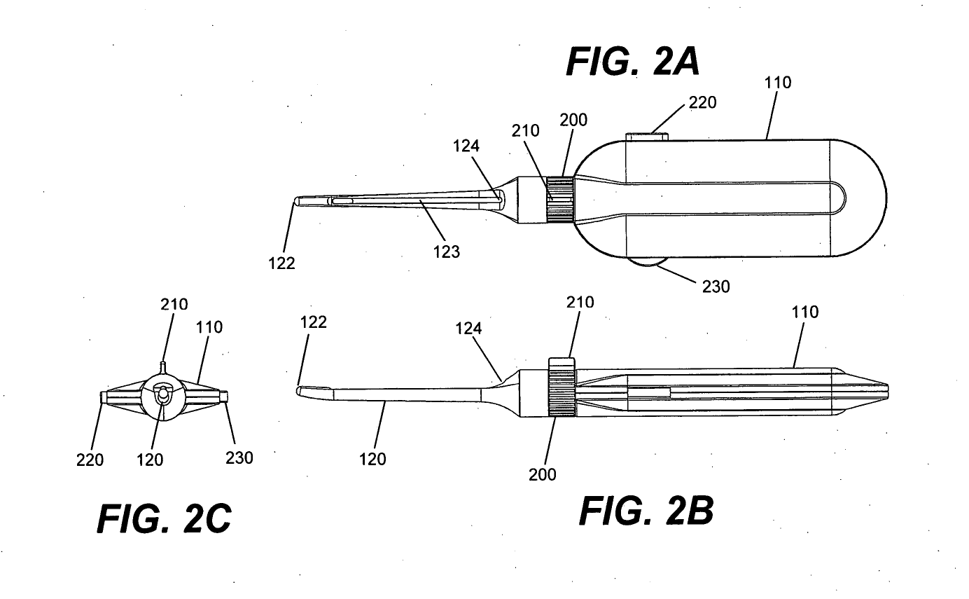

FIG. 2A shows a top view of an embodiment of the device, showing a cannula 120 attached at its proximal end to the distal end of a housing 110. In this embodiment, the cannula 120 comprises a longitudinal slot 123 that extends longitudinally from a proximal end 124 to near the distal end 122 of the cannula 120. In some embodiments, the proximal end 124 of the slot is raised to better allow tools to enter the slot without scraping against the proximal end 124. In some embodiments, at the junction of the housing 110 and the cannula 120, the device comprises a connecting ring 200 that attaches the cannula 120 to the housing 110. In some embodiments, the connecting ring 200 comprises a paddle 210 for ease of manipulation, such as when the device is being held and controlled within one hand. In some embodiments, the connecting ring allows for the interchangeable attachment of different types of cannulas 120 to a given housing 110, dependent upon the procedure to be performed with the device. In some embodiments, the housing 110 further comprises a rocker switch 220 that is used to move the selected tools within the housing or into and out of the cannula 120. In other embodiments, the movement of tools into and out of the cannula 120 is controlled electronically and/or remotely. In some embodiments, the housing 110 also comprises a switch 230 for turning the light on and off.

FIG. 2B is a side view of the embodiment of the device shown in FIG. 2A, showing the housing 110, cannula 120, and the intervening connecting ring 200 with paddle 210. In some embodiments, the distal end 122 of the cannula 120 is closed and turned upwards, serving as an integral obturator.

FIG. 2C is a distal end view of the embodiment of the device shown in FIG. 2A, showing the relative positions of the cannula 120, housing 110, paddle 210, slider knob 220 and switch 230.

### Kit

Another aspect of the present application relates to an instrument kit for implementing an endoscopic or arthroscopic surgical procedure. The kit comprises the video assisted surgical device of the present application.

In some embodiments, the instrument kit comprises additional components and implements useful for endoscopic or arthroscopic procedures.

In another embodiment, the instrument kit further includes a scalpel.

In another embodiment, the instrument kit further includes at least one retractor for holding open an entry portal.

In another embodiment, the instrument kit comprises a power source for the video assisted surgical device of the present application. In a further embodiment, the power source is a battery.

In another embodiment, the instrument kit comprises multiple interchangeable cannulas that can be interchangeably attached to the distal end of the device housing included in the kit. In some embodiments, at least one of said interchangeable cannulas comprises a longitudinal slot that extends from the proximal end to the proximity of the distal end. In some embodiments, at least one of said interchangeable cannulas comprises an open distal end. In some embodiments, at least one of said interchangeable cannulas comprises a longitudinal slot in the proximity of the distal end. In some embodiments, at least one of said interchangeable cannulas is a tube having an open distal end.

### Method for Endoscopic or Arthroscopic Surgery

Another aspect of the present application relates to a method for uniportal endoscopic or arthroscopic surgery using the video assisted surgical device of the present application. Uniportal endoscopic or arthroscopic surgery allows the practitioner to visualize a target tissue and its surrounding tissues as well as perform a surgical procedure through a single entry portal. In some instances, the entry portal may be a natural opening, while in other instances the entry portal is an incision. In the case of an incision, generally only a single small incision must be made. In particular embodiments, the incision is less than or equal to about 2 cm in length. In more particular embodiments, the incision is less than or equal to about 1.5 cm in length. In still more particular embodiments, the incision is less than or equal to about 1 cm in length. The single small incision allows the patient to recover more quickly and begin therapy and/or resume normal activity as tolerated sooner. In some embodiments, the procedure can be a uniportal percutaneous endoscopic surgical procedure.

The uniportal endoscopic surgical procedure described herein can be used to implement a number of different surgical procedures including, but not limited to, carpal tunnel release, Guyon's canal (or canal) release, cubital tunnel release, plantar fascia release, lateral release for patella realignment, release of radial tunnel, release of pronatar teres, release of trigger finger, release of lacertus fibrosus, release of the extensor tendons for lateral epicondylitis, release of medial epicondylitis, release of the posterior and other compartments of the leg, forearm fascia release for fascial compartment syndrome, release of fascial compartments in the upper or lower extremities, relieving the compression of a nerve by a ligament pulley or tunnel, releasing the travel of a ligament through a ligament pulley or tunnel, surgical procedures on the spine, such as endoscopic discectomy for the treatment of degenerative disc disease, herniated discs, bulging discs, pinched nerves or sciatica, endoscopic procedures on cranial and facial tissues, fasciotomy release and blood vessel harvesting.

One embodiment of the present application relates to a method for a performing a uniportal endoscopic or arthroscopic surgical procedure a target tissue in a subject. Generally, the surgical procedure requires the establishment of an entry portal. In some embodiments of the present application, the entry portal is established to the proximate side of the target tissue. In other embodiments of the present application, the entry portal is established to the distal side of the target tissue.

In some embodiments, the establishing an entry portal comprises making an incision.

In some embodiments, following the establishment of an entry portal, the distal end of the cannula portion of the device is inserted through the portal to establish an opening in the underlying tissue between the portal and the target tissue. In some embodiments, the distal end of the cannula portion of the device comprises a front edge for separating tissues. In some embodiments, the cannula is expanded laterally in order to spread the tissue, establishing a passage from the entry portal to the target tissue.

The camera is advanced from the housing and into the cannula to view the target tissue and the surrounding tissues, assuring that the slot of the cannula is in proper orientation to the target tissue.

In one embodiment, the surgical procedure is carpal tunnel release. In a further embodiment, the target tissue is the transverse carpal ligament.

In another particular embodiment, the surgical procedure is trigger finger release. In another particular embodiment, the target tissue is the A1 pulley.

### OTHER EMBODIMENTS

One aspect of the present application related to a device for wireless endoscopic and arthroscopic surgery. The device comprises a housing comprising a receptacle for a cannula; a camera with a lens; a control board having transmitter and receiver functions for wirelessly communicating with at least one external receiver/transmitter; an antenna; and a power source. The camera, the control board, the antenna and the power source are enclosed by the housing.

In some embodiments, the device can be handheld. In some further embodiments, the housing is formed in a shape to facilitate gripping with a single hand.

In other embodiments, the device can be held by any other device.

The device can be used on any target tissue, bone, joint or target area of the body of a subject.

In some embodiments, the device further comprises a clear cannula mounted in the receptacle of the housing, wherein the cannula comprises a proximal end, a distal end and an open slot extending from the proximal end to the proximity of the distal end.

In some further embodiments, the distal end of the cannula comprises an edge for separating tissues.

In some other embodiments, the distal end of the cannula is turned upwards like an obturator.

In still other embodiments, the device further comprises a blade and at least one of (1) an image transmitting optical fiber attached at its proximal end to the camera, wherein the distal end of the image transmitting optical fiber is movable into the cannula independently or with tools of the device, (2) a probe, (3) a cautery, and (4) a tool for grasping tissue or biopsy samples, wherein each of the blade, the probe, the cautery and the tool is located either at the distal end of the cannula, or is located inside the housing and extendable to a position in the proximity of the distal end of the cannula.

In some embodiments, the lens of the camera has a diameter of less than 5 mm.

In other embodiments, the light source is an LED light source.

In still other embodiments, the camera is a NANEYE camera.

Another aspect of the present application relates to a wireless device for performing video-assisted endoscopic and arthroscopic procedures, comprising: a cannula having a tubular body with a proximal end, a distal end and an open slot on the tubular body; a camera with a lens; a light source that illuminates into and through the cannula; a control board having transmitter and receiver functions for wirelessly communicating with at least one external receiver/transmitter; an antenna; and a power source, wherein the light source, the control board and the antenna are mounted inside an enclosed housing, wherein the housing has a distal end and a proximal end and wherein the cannula is attached to the distal end of the housing. In some embodiments, the device can be handheld. In some further embodiments, the housing is formed in a shape to facilitate gripping with a single hand.

In other embodiments, the device can be held by any other device.

The device can be used on any target tissue, bone, joint or target area of the body of a subject.

In some embodiments, the wireless device further comprises a blade and at least one of (1) an image transmitting optical fiber attached at its proximal end to the camera, wherein the distal end of the image transmitting optical fiber is movable into the cannula independently or with tools of the device, (2) a probe, (3) a cautery, and (4) a tool for grasping tissue or biopsy samples, wherein each of the blade, the probe, the cautery and the tool is located either at the distal end of the cannula, or is located inside the housing and extendable to a position in the proximity of the distal end of the cannula.

In other embodiments, the cannula comprises an open proximal end and an open distal end. In some further embodiments, the cannula comprises an open slot that extends from the open proximal end to the open distal end.

In still other embodiments, the cannula is flexible.

Yet another aspect of the present application relates to a system for wireless endoscopic and arthroscopic surgery. The system comprises a device for wireless endoscopic and arthroscopic surgery that comprises a housing comprising a receptacle for a cannula; a camera with a lens; a control board having transmitter and receiver functions for wirelessly communicating with at least one external receiver/transmitter; an antenna; and a power source, wherein the camera, the control board, the antenna and the power source are enclosed by the housing. The system further comprises an external receiver/transmitter for visualizing images transmitted by the device.

In some embodiments, the device can be handheld. In some further embodiments, the housing is formed in a shape to facilitate gripping with a single hand.

In other embodiments, the device can be held by any other device.

The device and system can be used on any target tissue, bone, joint or target area of the body of a subject.

In some embodiments, the system further comprises a clear cannula mounted in the receptacle of the housing, wherein the cannula comprises a proximal end, a distal end and an open slot extending from the proximal end to the proximity of the distal end.

In other embodiments of the system, the device further comprises at least one of (1) an image transmitting optical fiber attached at its proximal end to the camera, wherein the distal end of the image transmitting optical fiber is movable into the cannula independently or with tools of the device, (2) a probe, (3) a cautery, and (4) a tool for grasping tissue or biopsy samples that is extendable into the cannula and capable of protruding through the open slot to contact tissue.

In still other embodiments of the system, the external wireless receiver/transmitter is selected from the group consisting of video monitors, computer terminals comprising a monitor, smart phones and tablet computers. In some embodiments, function of the tools in the device is controlled with a dedicated application (app) installed or resident on the external wireless receiver/transmitter.

A further aspect of the present application relates to a method for performing a uniportal endoscopic or arthroscopic procedure with a wireless device for performing video-assisted endoscopic and arthroscopic surgical procedures, the device comprising: a cannula having a tubular body with a proximal end, a distal end and an open slot on the tubular body; a camera with a lens; a light source that illuminates into and through the cannula; a control board having transmitter and receiver functions for wirelessly communicating with at least one external receiver/transmitter; an antenna; and a power source, wherein the light source, the control board and the antenna are mounted inside an enclosed housing, wherein the housing has a distal end and a proximal end and wherein the cannula is attached to the distal end of the housing. The method comprises the steps of a) establishing an entry portal; b) inserting the distal end of the cannula through the entry portal; c) advancing the cannula to create a passage to the target tissue; d) imaging the target tissue with the camera; e) performing an endoscopic operation at the target tissue; and f) withdrawing the cannula from the entry portal. In some embodiments, the procedure can be a uniportal percutaneous endoscopic surgical procedure.

In some embodiments, the device can be handheld. In some further embodiments, the housing is formed in a shape to facilitate gripping with a single hand.

In other embodiments, the device can be held by any other device.

The device can be used on any target tissue, bone, joint or target area of the body of a subject.

In some embodiments, the endoscopic or arthroscopic procedure is a surgical operation comprising the steps of contacting the target tissue with a blade and dividing the target tissue with the blade. In some further embodiments, the endoscopic procedure is carpal tunnel release and the target tissue is the transverse carpal ligament. In other further embodiments, the endoscopic procedure is trigger finger release and the target tissue is the A1 pulley. In still other embodiments, the endoscopic or arthroscopic procedure is fascia release.

In other embodiments, the endoscopic or arthroscopic procedure is a biopsy comprising the steps of contacting the target tissue with a tool for grasping tissue or biopsy samples and retrieving a sample of the target tissue with the tool. In some further embodiments, the procedure comprises the further step of contacting the target tissue with the cautery and cauterizing the site the sample was retrieved from. In other further embodiments, the target tissue is a tumor.

Still another aspect of the present application relates to a kit for performing an endoscopic or arthroscopic procedure. The kit comprises a device for wireless endoscopic and arthroscopic surgery that comprises a housing comprising a receptacle for a cannula; a camera with a lens; a control board having transmitter and receiver functions for wirelessly communicating with at least one external receiver/transmitter; an antenna; and a power source, wherein the camera, the control board, the antenna and the power source are enclosed by the housing. The kit further comprises a clear cannula mountable to the device.

In some embodiments, the device can be handheld. In some further embodiments, the housing is formed in a shape to facilitate gripping with a single hand.

In other embodiments, the device can be held by any other device.

The device can be used on any target tissue, bone, joint or target area of the body of a subject.

The present invention is further illustrated by the following examples which should not be construed as limiting. The contents of all references, patents and published patent applications cited throughout this application, as well as the Figures, are incorporated herein by reference.

### Example 1

### Uniportal Endoscopic Carpal Tunnel Release

In a patient presenting with carpal tunnel syndrome, an incision is made just proximal or distal to the carpal transverse ligament, making an entry portal.

The distal end of the cannula of the video assisted surgical device is inserted into entry portal and the front edge of the cannula is introduced into the incision and used to create a plane under the carpal transverse ligament, but superficial to the median nerve, with the slot of the cannula facing the carpal transverse ligament. The procedure is observed with the camera. The camera is moved within the cannula to observe and image the target tissue and surrounding tissues.

Following the creation of the plane, the camera is withdrawn back into the housing of the device. The probe along with the camera is then advanced into the cannula to visualize the edges of the transverse carpal ligament. The probe and camera are then withdrawn back into the device.

The blade is then selected and in association with the camera and moved into the cannula. The cutting edge of the blade is moved into contact with the edge of the transverse carpal ligament and the ligament is divided by advancing the cutting edge of the blade through the ligament. The blade is retracted back into the housing of the device.

The camera is moved back into the cannula and the cut edges of the carpal transverse ligament and the underlying median nerve and tendons attached to the digits are visualized.

While visualizing the nerve and tendons, release is confirmed by passive manipulation of the digits through their range of motion.

The cannula is removed from the incision. The device is properly discarded as medical waste.

The wound is closed and a soft bandage is applied. In some cases, a splint is also applied to immobilize the wrist up to a week.

### Example 2

### Uniportal Endoscopic Trigger Release

In a patient presenting with trigger finger of the middle or ring finger, an incision is made just proximal to the A1 pulley on the distal palmar crease proximate to the affected digit or distal to the A1 pulley at or near the base of the affected digit.

The distal end of the cannula of the video assisted surgical device is inserted into entry portal and the front edge of the cannula is introduced into the incision and used to create a plane superficial to the flexor tendon sheath, with the slot of the cannula facing the flexor tendon sheath. The procedure is viewed through the camera. The camera is moved within the cannula to observe and image the target tissue and surrounding tissues.

Following the creation of the plane, the camera is withdrawn back into the housing of the device. The probe along with the camera is then advanced into the cannula to visualize the edges of the flexor tendon sheath. The probe and camera are then withdrawn back into the device.

The blade is then selected and in association with the camera and moved into the cannula. The cutting edge of the blade is moved into contact with the edge of the flexor tendon sheath and the sheath is divided by advancing the cutting edge of the blade through the ligament. The blade is refracted back into the housing of the device.

The camera is moved back into the cannula and the cut edges of the flexor tendon sheath and the underlying flexor tendon and tendons attached to the digits are visualized. While visualizing the tendon, release of the tendon is confirmed by passive manipulation of the digit through its range of motion.

The wound is closed and a soft bandage is applied.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The aspects and embodiments are intended to cover the components and steps in any sequence which is effective to meet the objectives there intended, unless the context specifically indicates the contrary.

What is claimed is:

1. A device for wireless endoscopic and arthroscopic surgery, comprising:
a housing comprising a receptacle for a cannula;
a camera with a lens;
a control board having transmitter and receiver functions for wirelessly communicating with at least one external receiver/transmitter;
an antenna; and
a power source,
wherein said control board, antenna and power source are enclosed by the housing.

2. The device of claim 1, wherein said device can be used on any target tissue, bone, joint or target area of the body of a subject.

3. The device of claim 1, wherein said device can be handheld.

4. The device of claim 1, wherein said camera is enclosed in the housing.

5. The device of claim 1, wherein said camera is extendable into the cannula.

6. The device of claim 1, further comprising a clear cannula mounted in the receptacle of said housing, wherein said cannula comprises a proximal end, a distal end and an open slot extending from the proximal end to the proximity of the distal end.

7. The device of claim 6, wherein said distal end of the cannula comprises an edge for separating tissues.

8. The device of claim 6, wherein said distal end of the cannula is turned upwards like an obturator.

9. The device of claim 6, wherein said device further comprises a blade and at least one of (1) an image transmitting optical fiber attached at its proximal end to the camera, wherein the distal end of the image transmitting optical fiber is movable into the cannula independently or with tools of the device, (2) a probe, (3) a cautery, and (4) a tool for grasping tissue or biopsy samples, wherein each of the blade, the probe, the cautery and the tool is located either at the distal end of the cannula, or is located inside the housing and extendable to a position in the proximity of the distal end of the cannula.

10. The device of claim 1, wherein the lens of said camera has a diameter of less than 5 mm.

11. The device of claim 1, wherein said light source is an LED light source.

12. The device of claim 1, wherein said camera is a NANEYE camera.

13. A wireless device for performing video-assisted endoscopic and arthroscopic surgical procedures, comprising:
a cannula having a tubular body with a proximal end, a distal end and an open slot on the tubular body;
a camera with a lens;
a light source that illuminates into and through the cannula;
a control board having transmitter and receiver functions for wirelessly communicating with at least one external receiver/transmitter;
an antenna; and
a power source,
wherein said light source, control board and antenna are mounted inside an enclosed housing, wherein said housing has a distal end and a proximal end and wherein the cannula is attached to the distal end of the housing.

14. The wireless device of claim 1, wherein said device can be used on any target tissue, bone, joint or target area of the body of a subject.

15. The wireless device of claim 13, wherein said device can be handheld.

16. The device of claim 13, wherein said camera is enclosed in the housing.

17. The device of claim 13, wherein said camera is extendable into the cannula.

18. The wireless device of claim 13, further comprising a blade and at least one of (1) an image transmitting optical fiber attached at its proximal end to the camera, wherein the distal end of the image transmitting optical fiber is movable into the cannula independently or with tools of the device, (2) a probe, (3) a cautery, and (4) a tool for grasping tissue or biopsy samples, wherein each of the blade, the probe, the cautery and the tool is located either at the distal end of the cannula, or is located inside the housing and extendable to a position in the proximity of the distal end of the cannula.

19. The wireless device of claim 13, wherein said cannula comprises an open proximal end and an open distal end, and wherein said open slot extends from said open proximal end to the open distal end.

20. The wireless device of claim 13, wherein the cannula is flexible.

21. A system for wireless endoscopic and arthroscopic surgery, comprising:
the device of claim 1; and
an external receiver/transmitter for visualizing images transmitted by said device.

22. The system of claim 21, wherein said device can be used on any target tissue, bone, joint or target area of the body of a subject.

23. The system of claim 21, further comprising a clear cannula mounted in the receptacle of the housing, wherein the cannula comprises a proximal end, a distal end and an open slot extending from the proximal end to the proximity of the distal end.

24. The system of claim 23, wherein the device further comprises at least one of (1) an image transmitting optical fiber attached at its proximal end to the camera, wherein the distal end of the image transmitting optical fiber is movable into the cannula independently or with tools of the device, (2) a probe, (3) a cautery, and (4) a tool for grasping tissue or biopsy samples that is extendable into the cannula and capable of protruding through the open slot to contact tissue.

25. The system of claim 21, wherein the external wireless receiver/transmitter is selected from the group consisting of video monitors, computer terminals comprising a monitor, smart phones and tablet computers.

26. A method for performing a uniportal endoscopic or arthroscopic procedure with the wireless device of claim 13, comprising:
a) establishing an entry portal;
b) inserting the distal end of the cannula through the entry portal;
c) advancing the cannula to create a passage to the target tissue;
d) imaging the target tissue with the camera;
e) performing an endoscopic operation at the target tissue; and
f) withdrawing the cannula from the entry portal.

27. The method of claim 26, wherein said device can be used on any target tissue, bone, joint or target area of the body of a subject.

28. The method of claim 26, wherein said endoscopic or arthroscopic procedure is a surgical operation comprising the steps of contacting the target tissue with a blade and dividing the target tissue with the blade.

29. The method of claim 28, wherein said endoscopic procedure is carpal tunnel release and the target tissue is the transverse carpal ligament.

30. The method of claim 28, wherein said endoscopic procedure is trigger finger release and the target tissue is the A1 pulley.

31. The method of claim 26, wherein said endoscopic or arthroscopic procedure is a biopsy comprising the steps of contacting the target tissue with a tool for grasping tissue or biopsy samples and retrieving a sample of said target tissue with the tool.

32. The method of claim 31, comprising the further step of contacting said target tissue with the cautery and cauterizing the site of said sample was retrieved from.

33. The method of claim 31, wherein said target tissue is a tumor.

34. The method of claim 28, wherein said endoscopic or arthroscopic procedure is fascia release.

35. A kit for performing an endoscopic or arthroscopic procedure, comprising:
the device of claim 1; and
a clear cannula mountable to said device.

36. The kit of claim 35, wherein said device can be used on any target tissue, bone, joint or target area of the body of a subject.

37. The device of claim 2, wherein said device can be used in a procedure including the head, neck, chest, abdomen, upper extremity or lower extremity.

38. The device of claim 37, wherein said device can be used in a procedure including a finger, hand, foot of said subject.

39. The device of claim 37, wherein said device can be used in plastic surgery.
